(19) United States
(12) Patent Application Publication
(10) Pub. No.: US 2005/0080040 A1
(43) Pub. Date: Apr. 14, 2005

(54) Treatment of tissue, instruments and work surfaces to remove infectious agents

(76) Inventors: Crystal M. Cunanan, Mission Viejo, CA (US); Tan Thanh Dinh, Fountain Valley, CA (US); Christine Loshbaugh, Irvine, CA (US); H. Chris Sarner, Laguna Hills, CA (US); Michael N. Helmus, Worcester, MA (US)

Correspondence Address: John Christopher James;Edwards Lifesciences LLC, Law Dept., One Edwards Way, Irvine, CA, 92614, US

(21) Appl. No.: 10/959,549

(22) Filed: Oct. 5, 2004

Related U.S. Application Data
(62) Division of application No. 09/930,619, filed on Aug. 15, 2001, now abandoned.

(51) Int. Cl. A61K 31/737; A61K 31/727

(52) U.S. Cl. 514/54; 514/56; 514/59

(57) ABSTRACT

The present invention provides methods of inactivating and removing infectious agents from tissues of use in bioprosthetic devices. The methods include the removal and blockage of binding sites on the tissues for the infectious agents. Also provided are methods for blocking a site on an infectious agent that binds to a site on the tissue.

### RELATED APPLICATION DATA

This application is a divisional of U.S. patent application Ser. No. 09/930,619, the entire disclosure of which is incorporated herein by reference.

### BACKGROUND OF THE INVENTION

Implantable xenografts and prosthetic devices are typically sterilized prior to implantation in an intended recipient. Sterilization is required to ensure that the devices do not introduce potential pathogens, or other biologically detrimental agents into the intended recipient. Sterilization is particularly relevant where biomaterials from human or other mammalian donors are constituents of the graft or device. Examples of biological tissues that have been used to form implantable bioprostheses include cardiac valves, blood vessels, skin, dura mater, pericardium, ligaments, and tendons.

Device components are sterilized individually prior to assembling the device or, alternatively, they are sterilized by the process of “terminal sterilization”. In the terminal sterilization process, the device is sterilized following its construction, i.e., after all the components have been combined with one another in the device. Both processes may be used in combination to ensure complete sterilization of the graft or device. A variety of physical or chemical methods have been developed for use in sterilization and include, for example, exposure to chemicals or heat, or exposure to ionizing or non-ionizing radiation.

Historically, for infectious agents such as bacteria, there are well established methods of control that involve different forms of sterilization, for example, steam sterilization, dry sterilization, pasteurization, sterile filtration, radiation inactivation, and the like. With viruses, there are also established methods which involve lowering of the pH to 4.0 or below, or use of organic solvents in high concentrations. Extended periods of heating at 60° C. also may be used. In addition, UV radiation treatment, formaldehyde and specific antiviral agents have been employed to mitigate the potential harm associated with viruses.

These methods, however, have inherent problems and may have adverse effects on biological tissue. Consequently, most of the methods are inappropriate for bioprosthetic devices incorporating mammalian tissue. Further, many of the methods are not effective for preventing or inhibiting associated with certain classes of infectious agents, such as prions.

Exemplary sterilization methods include treating prosthesis and graft components with chemical reagents. The chemical reagents themselves, or reaction byproducts derived from the reagents, can be harmful to the intended recipient of the prosthetic device. Accordingly, such chemicals must be removed prior to implantation of the devices. Common chemical sterilizing agents include ethylene oxide and formaldehyde, both of which are alkylating agents and, therefore, can modify and inactivate biologically active molecules. See, Davis et al., (1973) “Microbiology, 2nd Ed.”, Harper and Row, Publishers.

Other methods of sterilizing device components include exposing the device or components thereof to heat, ionizing radiation, or plasma. See, Moulton et al., U.S. Pat. No. 5,084,239. Exposing a device, which includes biological components (e.g. proteins, cells, tissues), to elevated temperatures, radiation, or plasma is not desirable because proteins and other biological materials can be denatured and subsequently inactivated or weakened by exposure to these forms of energy. Although the sterilization of objects by exposure to ionizing and non-ionizing radiation obviates the necessity of adding potentially toxic chemicals, the radiation energy and/or its byproducts, including oxygen free radicals, are competent to modify protein conformation and so can damage or destroy proteins, cells, and tissue. In addition, exposure of some medically important polymers, for example, polyurethane or polymethylmethacrylate to gamma radiation can result in immediate and long term physical changes to the polymer. Moreover, irradiation with gamma or beta rays does not destroy all pathogens with certainty. Indeed, certain viruses are radiation resistant. Thus, alternatives to sterilizing xenografts and biologically derived components of prosthetic devices with harsh chemicals and radiation are being avidly sought.

A number of infectious agents pose a threat to the safety of implantable bioprosthetic devices. Among these are bacteria, viruses, retroviruses, fungi, mycoplasma, and prions. Various proteins and nucleic acids may also act as infectious agents. Of increasing concern is the presence of infectious prions in biologically derived materials used for xenografts and prosthetic devices. The widespread occurrence of prion-related disease and the possibility of interspecies transmission has serious implications for the biotechnology industry, which derives many of its products from mammalian tissue. See, Di Martino Biologicals 21: 61-66 (1993). Concerns about the safety of mammalian tissue products has led to studies on the inactivation of prions. These studies indicate that prions are more resistant toward inactivation than more conventional pathogens such as viruses or bacteria. Thus, relatively harsh conditions are required to decontaminate prion-containing biological materials. The only methods currently known to disinfect prion contaminated biological preparations are prolonged autoclaving at 130° C. or above, and treatment with concentrated sodium hydroxide solution. These methods have been recommended for routine inactivation of prions. See, Department of Health and Social Security Circular 84: 16 (1989). It has also been reported that 100 kD cutoff ultrafiltration in combination with treatment with 6M urea results in decontamination of prion containing preparations. See, Pocchiari et al., Arch. Virol. 98: 131-135 (1988). Other methods capable of lowering prion activity include treatment with organic solvents, detergents, protein-denaturing agents, chaotropic salts and phenol. See, Millson et al., in Prusiner and Hadlow, eds. SLOW TRANSMISSIBLE DISEASES OF THE NERVOUS SYSTEM, vol. II. New York: Academic Press 409-424 (1979); Prusiner et al., Proc. Natl. Acad. Sci. USA 78: 4606-4610 (1981); Kimberlin et al., J. Neurol. Sci. 59: 390-392 (1983); Walker et al., Am. J. Public Health 73: 661-665 (1983); and Brown et al., J. Infect. Dis. 153: 1145-1148 (1986). Each of the above-recited methods is generally inappropriate for treating tissues.

Additional methods of removing prions from biological material include removing prions from solution by directing the solution through an anion-exchange chromatography column. Gawryl et al., in U.S. Pat. No. 5,808,011. Filtration methods, however, are not suitable for treating whole tissues, where the structural integrity of the tissue membrane must be preserved.

In U.S. Pat. No. 5,780,288, Rohwer et al. describe a method for destroying infectivity in a proteinaceous mixture such as animal feed. This method involves the application of harsh alkali and heat treatments, and while this may be effective for disinfecting animal feed, the method is not appropriate for treating bioprosthetic tissue.

In U.S. Pat. No. 5,756,678, Shenoy et al. describe a method for treating solubilized collagen to inactivate prions and other infective agents, via the application of sodium hydroxide. Shenoy et al. do not, however, suggest that this method would be effective for treating bioprosthetic tissue.

Cashman et al., in PCT publications WO 97/45746, WO 00/78344, and WO 01/00235, discuss methods for treating prion infectivity in biological tissue that include contacting the tissue with prion binding proteins such as protocadherins and antibodies. methods making use of other compounds to remove or inactivate infectious materials in tissues are not disclosed.

Reichl, in U.S. Pat. No. 5,633,349 describes a method for inactivating prions and other infectious agents in plasma by contacting the plasma with a chaotropic agent such as urea or sodium thiocyanate. Reichl does not discuss a method for treating bioprosthetic tissue designed as a transplant graft.

In U.S. Pat. No. 6,150,172, Alpert et al. discuss a method for extracting infectious prion from a biological material using an extraction solvent such as a polar organic solvent. Alpert et al. do not describe a method for treating bioprosthetic tissue for a transplant graft.

Narotam et al., in U.S. Pat. No. 5,997,895, and Doillon et al, in U.S. Pat. No. 6,197,935, describe methods for rendering collagen preparations free from viral and prion infectivity. As these methods involve intensive mechanical disruption of the collagen material, they would not be suitable for treating bioprosthetic tissue as contemplated by the present invention.

Prusiner et al., in WO 00/43782, discuss a method for removing prion from a liquid sample. As described, the liquid sample is flowed across a solid surface that contains a prion complexing agent. Removal methods such as these are not suitable for treating whole tissue, where the structural integrity of the tissue membrane must be preserved.

A recent survey of prion inactivation methods discusses approaches such as moist heat (autoclaving), filtration, formaldehyde, heat, phenols, irradiation, potassium permanganate, sodium dodecyl sulfate, sodium hydroxide, sodium perchlorate, and sodium hypochlorite. The survey indicates that while several avenues are being investigated, the search continues for a process that can substantially minimize the risk of infection. See, Cox, Institute for International Research Conference, Mar. 15-16, 1999, San Diego.

The extreme conditions required to eliminate infectivity, and particularly prion infectivity, in biomaterials are typically incompatible with methods intended to preserve the useful activity and structure of these materials. The harsh conditions of prior methods are particularly deleterious to mammalian tissue, resulting in the denaturation of functional and structural components of the tissues. Thus, there is a need for a method for treating or preventing infectivity in mammalian tissue that does not compromise the integrity of these desirable biomaterials. The present invention meets these and other needs.

### BRIEF SUMMARY OF THE INVENTION

There has been a recognized absence, in the tissue treatment arts, of mild chemical and enzymatic methods for preventing or reducing infectivity in bioprosthetic tissues. The present invention provides an effective protocol for preparing mammalian tissue for incorporation into a bioprosthetic device. The invention is based on the discovery that mild chemical or enzymatic treatments are effective at eliminating or inhibiting a surprisingly high level of infectivity from tissue or biological samples. Further, these procedures do not significantly degrade or denature tissue proteins, and thus tissues prepared under this protocol are well suited for use in bioprosthetic devices.

The methods of the present invention are effective, not only for preventing infectious agents from binding to the tissue, but also for removing infectious agents from tissue under conditions that are notably more mild than previous art-recognized methods. Although the methods described herein are of general applicability, the present invention particularly concerns a method for reducing or eliminating infectious agent contamination in bioprosthetic tissue preparations.

Thus, in a first aspect, the present invention provides a method of eliminating or reducing infection in a bioprosthetic tissue. Tissue cells typically have binding sites on their surface that can be recognized by infectious agents. Binding sites may also be recognized by other unwanted substances, such as certain enzymes, proteins, protein precursors, and the like. Often, these binding sites contain phospholipid components. Therefore, the method includes removing phospholipid binding sites located in the tissue, so that infectious agents or other unwanted substances are prevented or inhibited from binding to the tissue.

In a second aspect, the present invention provides a method of eliminating or reducing infection in a bioprosthetic tissue. While binding sites may contain phospholipid components, they may optionally or also contain protein or polysaccharide moieties. Therefore, the method also includes removing protein and/or polysaccharide binding sites located in the tissue, so that infectious agents or other unwanted substances are prevented or inhibited from binding to the tissue.

In a third aspect, the present invention provides a method of eliminating or reducing infection in a bioprosthetic tissue. The method includes blocking an infectious agent binding site contained in the tissue, so that infectious agents are prevented or inhibited from binding to the tissue.

In a fourth aspect, the present invention provides a method of eliminating or reducing infection in a bioprosthetic tissue. The method comprises contacting the tissue with a preparation having binding affinity for the infectious agent, thereby blocking the agent from infecting or contaminating the tissue. Thereafter, the binding agent may be optionally washed from the tissue in an extraction procedure, thereby removing the agent from the tissue.

The artisan will appreciate that in addition to treating bioprosthetic tissues, the methods of the present invention are well suited for disinfecting, sterilizing, decontaminating, or otherwise preventing contamination in a wide variety of medical instruments and laboratory work surfaces.

Other objects and advantages of the present invention will be apparent from the detailed description that follows.

### DETAILED DESCRIPTION OF THE INVENTION AND THE PREFERRED EMBODIMENTS

Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

As used herein, the term “remove” refers to a process of purging a selected agent from a tissue or other biological sample. Typically, this means eliminating a binding site or an infectious agent from a bioprosthetic tissue.

The term “block” refers to a process of inhibiting or eliminating any propensity of association between an infectious agent and a corresponding binding site. This is generally accomplished by the administration of a blocking agent. For example, blocking a particular infectious agent may render it unable to adhere to a complementary binding site. Alternatively, blocking a particular binding site may render it unable to adhere to an associated infectious agent. Generally, it is the physical presence of the blocking agent that interferes with the association between the infectious agent and the binding site. In some instances, a blocking agent will competitively bind to a selected binding site of a biological material.

While a blocking agent may physically or chemically obstruct association between the binding site and the infectious agent, the invention also contemplates other mechanisms of action. For example, the blocking agent may also or optionally act to modify or otherwise change the binding site, the infectious agent, or both, so that association between the two is weakened or eliminated. Accordingly, the continued presence of the blocking agent may or may not be required to obstruct or interfere with the association.

“Infectious agent” refers to an agent targeted by the process of the invention, and include, without limitation, viruses, bacteria, mycobacteria, mycoplasma, fungi, prions, prion pre-cursors, and constituents thereof. The term “infectious agent” also includes DNA, RNA, nucleic acids, proteins, peptides, amino acids, and carbohydrates. Further, this term refers to any molecule or composition of biological origin capable of serving as a causal agent of disease.

As used herein, the term “about” or “approximately” means within 20%, preferably within 10%, and more preferably within 5% of a given value or range.

Examples of “biological material” from human or animal include, but are by no means limited to, tissue such as brain, muscle (including heart), liver, appendix, pancreas, gastrointestinal tract organs, skin, bone, cartilage, tendon, ligament, connective tissue, and lymphoid tissue such as thymus, spleen, tonsil, lymph nodes, and the like. Alternatively, the biological material may be a biological fluid. The term biological fluid refers to cerebrospinal fluid, blood, serum, plasma, milk, urine, saliva, tears, mucous secretions, sweat, semen and bodily fluids comprising these components. It also refers to culture fluid (or culture medium) used in the production of recombinant proteins or containing cells in suspension prior to transplantation. Also encompassed by the term “biological materials” are products made from human or animal organs or tissues, including serum proteins (such as albumin and immunoglobulin), hormones, food and processed food products, nutritional supplements, bone meal, animal feed, extracellular matrix proteins, gelatin, and other human or animal by products used in manufacturing or final goods. The term additionally refers to any material that can be found in a human or animal that is susceptible to infection or that may carry or transmit infection.

The term “medical instrument” refers to a wide variety of devices used in surgical and medical procedures and in fabricating bioprosthetic devices. These include, but are not limited to, catheters, cannulas, dialysis or transfusion devices, shunts, stents, sutures, scissors, needles, stylets, devices for accessing the interior of the body, implantable ports, blades, scalpels. The term “medical instrument” is intended to encompass any type of device or apparatus that is used to contact a patient, and in particular used to contact the interior of the patient. The term also encompasses any device or tool used in the preparation or manufacture, or otherwise comes into contact with, a biological tissue.

“Laboratory work surface” refers to any work surface used by clinicians, surgeons, physicians, researchers, manufacturers, or anyone involved in the preparation, use, manufacture, or packaging of biological tissues or medical instruments that are intended for use in a patient. As work surfaces present a potential source of contamination, the methods of the present invention are well suited for treating these surfaces as well.

“Binding site” refers that portion of the biological material that forms an association with an infectious agent. For example, a binding site may include phospholipid, protein, and/or polysaccharide components. The term “binding site” also refers to that portion of the biological material that forms an association with certain enzymes, proteins, infectious agents, or other selected biomolecules.

The terms “prion,” “prion protein,” “PrP protein,” “PrP,” and like are used interchangeably herein and shall mean both the infectious particle form PrP^{Sc} known to cause diseases (spongiform encephalopathies) in humans and animals as well as the noninfectious form PrP^{C} which, under appropriate conditions, is converted to the infectious PrP^{Sc} form. The term prion will also refer to fragments or proteolytic digestion products of the complete form of the prion protein. The term prion is a contraction of the words “protein” and “infection.”

Infectious prion protein is much less susceptible to proteolysis than noninfectious prion protein. For example, treatment of a biological material with a proteinase, particularly proteinase-K, has been shown to digest noninfectious prion protein, but not infectious prion protein.

Prion protein is a native protein expressed in neural tissue, particularly the brain and at lower levels in lymphoid tissues and all other tissues. Prion protein generally occurs in PrP dimers, and is distinct from bacteria, viruses and viroids.

Typically, prions are encoded by a PrP gene. The terms “PrP gene,” “prion gene,” and the like are used interchangeably to describe genetic material which expresses proteins including known polymorphisms and pathogenic mutations thereof, it being recognized that the term includes other such PrP genes that are yet to be discovered. The term “PrP gene” refers generally to any gene of any species which encodes any form of a PrP protein. Some commonly known PrP sequences are described in Gabriel et al., Proc. Natl. Acad. Sci. USA 89:9097-9101 (1992); U.S. Pat. Nos. 5,565,186; 5,763,740; 5,792,901; and PCT Publication W097/04814, incorporated herein by reference to disclose and describe such sequences. The protein expressed by such a gene can assume either a PrP^{C} (non-disease) or PrP^{Sc} (disease) form. Certain mutations of the prion gene in some individuals appear to predispose prion protein to adopt the pathogenic conformation.

The term “causal agent” is intended to refer to an agent which either causes a disease or is a necessary component of a disease-producing system. Causal agents associated with a wide variety of diseases can be treated by the method of the present invention. For instance, the prion is a causal agent of several central nervous system diseases which are discussed below. The prion can be, for example, a protein, such as a post-translationally modified PrP protein, or it can be a protein complexed with an informational molecule, such as a polynucleotide, for example, a polydeoxy-ribonucleotide complexed with a post-translationally modified PrP protein.

“Prion disease” refers to one of several rapidly progressive, fatal, and untreatable brain degenerative disorders. These are generally considered to be transmissible spongiform encephalopathies (TSE), a group that includes, but without limitation: Creutzfeldt-Jakob disease (CJD), new variant CJD, Kuru, Gerstmann-Straussler-Scheinken syndrome (GSS), fatal familial insomnia (FFI) in humans, scrapie in sheep and goats, spongiform encephalopathy in cattle “mad cow disease”, as well as recently described prion diseases in cats, and other ruminants. Prion infection has also been observed in chicken, mink, pigs, mice, hamsters, guinea pigs, eland, elk, gemsbok, greater kudu, muledeer, nyala, oryx, and various avian species. Prion infection from these and other sources can be treated by the method of the present invention.

Generally, prion protein is found in vertebrates. Alternatively, prion protein can also be produced during fermentation processes with eukaryotic cells. It may be expressed as a recombinant prion protein. Of greater concern is the possibility of incidental expression of endogenous prion protein by cells that have been recombinantly modified to express another protein. This possibility is more likely if the cells are of neural origin, such as PC12 cells. In this case, the biological material may be a fermentation product, e.g., recombinant protein.

The phrase “substantially free of infectious agent” means that the product does not contain infection-effective amounts of infectious agent.

For the purposes of the present invention, the terms “surfactant” and “detergent” are used interchangeably and both shall refer to compounds or ions that (1) are made of groups of opposing solubility tendencies, such as an oil-soluble hydrocarbon chain and a water-soluble ionic group, (2) are soluble in at least one phase of a liquid system, (3) have a concentration at a phase interface that is greater than its concentration in the bulk of the solution when at equilibrium and in solute form, (4) form oriented monolayers at phase interfaces, (4) form micelle aggregates at the critical micelle concentration, and (5) exhibit combinations of cleaning, foaming, wetting, emulsifying, solubilizing, and dispersing properties when in solution. Perhaps the hallmark feature of a surfactant is the presence of two structurally dissimilar groups within a single molecule. Further defining features of surfactants can be found in KIRK-OTHMER'S “ENCYCLOPEDIA OF CHEMICAL TECHNOLOGY,” Third Edition, Vol. 22, pp. 332-432, which is hereby incorporated by reference.

The Methods

The present invention provides methods for removing or blocking a binding site present in mammalian tissue. The present invention also provides methods for removing or blocking an infectious agent or other protein associated with mammalian tissues.

In a first aspect, the present invention provides a method for eliminating or reducing infectivity in a biological material. A related embodiment provides a method for eliminating or reducing prion infection in a bioprosthetic tissue. The method includes removing a prion protein binding site contained in the tissue so that prion protein is prevented or inhibited from binding to the tissue. Typically, the invention is applicable to combating prion protein infection, and for preventing its transmission during tissue grafts. The invention is also applicable to combating a wide variety of bacteria including, but not limited to, Pseudomonas aeruginosa, Campylobacter upsaliensis, and Escherichia coli, and for preventing their transmission during implantation of tissue grafts and other bioprosthetic devices.

Bacteria have been implicated in various diseases. Often, phospholipids are implicated as the receptor binding sites recognized by these opportunistic pathogens. For example, two phospholipids in rabbit corneal epithelium, phosphatidylserine and phosphatidylinositol, provide bacterial binding sites. See, e.g., Panjwani et al., “Pathogenesis of Corneal Infection: Binding of Pseudomonas aeruginosa to Specific Phospholipids,” Infect. Immun. 64(5): 1819-1825 (1996). Phosphatidylethanolamine, gangliotetraosylceramide (Gg4), and phosphatidylserine have been shown to be exhibit binding activity to infectious agents. See, e.g., Sylvester et al., “Adherence to Lipids and Intestinal Mucin by a Recently Recognized Human Pathogen, Campylobacter upsaliensis,” Infect. Immun. 64(10): 4060-4066 (1996). Further, phosphatidylethanolamine is recognized as exhibiting binding activity to other bacterial pathogens. See, e.g., Foster et al., “Phosphatidylethanolamine Recognition Promotes Enteropathogenic E. coli and Enterohemorrhagic E. coli Host Cell Attachment,” Microb. Pathog. 27(5): 289-301 (1999).

Based on the foregoing, it is clear that phospholipid binding sites for infectious agents are well known in the art. In a preferred embodiment, the method of the present invention provides eliminating or inhibiting infectivity in a tissue by contacting the tissue with a composition that removes the phospholipid binding sites contained in the tissue.

In a related aspect, the present invention provides a method for eliminating or reducing the presence of various membrane-bound enzymes, proteins, or precursor proteins having a biological function that could adversely affect the performance and durability of a tissue derived bioprosthesis. For example, alkaline phosphatase activity has been linked to initiation of calcification in small animal models. Phospholipids have been shown to play a role in the binding of this enzyme to cell membranes. See, e.g., Low et al., “Role of Phosphatidylinositol in Attachment of Alkaline Phosphatase to Membranes,” Biochem. 19(17): 3913-3918 (1980). In a preferred embodiment, the present invention provides a method for treating a tissue by removing the phospholipid that functions as the binding site for this enzyme.

Likewise, the enzyme acetylcholinesterase is believed to bind to the plasma membrane via a phospholipid, particularly phosphatidylinositol. See, e.g., Futerman et al., “Identification of Covalently Bound Inositol in the Hydrophobic Membrane-Anchoring Domain of Torpedo acetylcholinesterase,” Biochem. Biophys. Res. Commun. 129(1): 312-317 (1985), and Cross, “Eukaryotic Protein Modification and Membrane Attachment Via Phosphatidylinositol,” Cell 48: 179-181 (1987). In a preferred embodiment, the present invention provides a method for treating tissue by removing the phospholipid that functions as the binding site for acetylcholinesterase.

Similarly, Thy-1 is a protein associated with immune cell recognition. Phospholipids have been shown to play a role in the binding of this protein. See, e.g., Low et al., “Phosphatidylinositol is the Membrane-Anchoring Domain of the Thy-1 Glycoprotein,” Nature 318(6041): 62-64 (1985). In a preferred embodiment, the present invention provides a method for treating a tissue by removing the phospholipid that functions as the binding site for Thy-1.

Similarly, prion protein is an infectious agent of considerable interest. Phospholipids are believed to play a role in the binding of this protein. See, for example, Di Martino et al., “The Consistent Use of Organic Solvents for Purification of Phospholipids from Brain Tissue Effectively Removes Scrapie Activity,” Biologicals 22(3):221-225 (1994). It is believed that prion is attached to the cell wall membrane via the phosphatidylinositol moiety of a glycosyl-phosphatidylinositol (GPI) molecule. In a preferred embodiment, the present invention provides a method for treating a tissue by removing the phospholipid that functions as the binding site for prion protein.

In a preferred embodiment, the tissue with which the present method is practiced includes substantially any mammalian tissue that is useful in preparing a prosthetic device having a biological component thereto. For example, in one embodiment, the tissue is derived from an organ. In another embodiment, the tissue is selected from nerve tissue, glandular tissue (e.g., lymphatic tissue), respiratory tissue, digestive tissue, urinary tract tissue, sensory tissue (e.g., cornea, lens, etc.), and reproductive tissue. In a related embodiment where the biological material is a biological fluid, however, addition of liquid is not likely to be necessary, unless to dilute the ionic strength of the biological fluid to permit miscibility of the extraction solvent.

In presently a preferred embodiment, the tissue is selected from muscle tissue, adipose tissue, epithelial tissue and endothelial tissue. In particularly preferred embodiments, the tissue is selected from myocardial tissue and vascular tissue. In a related embodiment, the tissue is selected from the group including, without limitation, heart valve, venous valve, blood vessel, ureter, tendon, dura mater, skin, pericardium, intestine (e.g., intestinal wall), or periostium. In a particularly preferred embodiment, the tissue is derived from bone, cartilage (e.g. meniscus), tendon, ligament, or any other connective tissue.

As the source of the material used for this purpose may vary with regard to both tissue type, the source may also vary with regard to species type (autologous, homologous or heterologous tissue). The artisan will appreciate that the methods of the present invention may be used with bioprosthetic devices that include one or more types of tissues or materials.

In a preferred embodiment where the biological material is a solid tissue or product, it may first be suspended in an aqueous solution so that it will be suitable for the extraction process. For example, brain tissue may be suspended in sucrose solution (e.g., 0.32 M sucrose) at 10% weight to volume. Other hypotonic or isotonic solutions include 5% dextrose, phosphate buffered saline, tri-buffered saline, HEPES-buffered saline, or any of the foregoing buffers. The biological material in the aqueous solution can also be homogenized, ground, or otherwise disrupted to maximize contact between the treatment agents and the biological material.

In a particularly preferred embodiment, the biological material will form part or all of a bioprosthetic tissue that is designed and intended for implantation into a graft recipient.

In a preferred embodiment, the infectious agent binding site includes one or more phospholipids. In related embodiments, the binding site is from the group including, without limitation, phosphatidic acid (PA), phosphoethanolamine, phosphotidylserine (PS), phosphotidylinositol (PI), phosphotidylcholine (PC), and sphingomyelin (SM). In further related embodiments, the binding site is from the group including phosphatidylethanolamine (PE), and gangliotetraosylceramide (Gg4).

In an exemplary embodiment, the method includes contacting the bioprosthetic tissue with a surfactant. In a particularly preferred embodiment, the surfactant is Tween 80. In another exemplary embodiment, the method includes contacting the bioprosthetic tissue with a preparation containing a surfactant, as well as a denaturing agent, such as a protic solvent. In a particularly preferred embodiment, the surfactant is Tween 80, and the protic solvent is ethanol or isopropanol. In related embodiment, the preparation further contains a cross linking agent, such as an aldehyde, and the aldehyde is preferably formaldehyde or glutaraldehyde.

Presently preferred aldehydes include both mono- and polyaldehydes. Aldehydes of use in practicing the present invention include any aldehyde, either substantially pure or containing additives, that prevents or inhibits infectivity in mammalian tissue. Although any aldehyde that has desirable characteristics for a particular application can be used to practice the present invention, certain aldehydes are presently preferred.

Preferred aldehydes include one or more compounds of the group consisting of acetaldehyde, butyraldehyde, isobutyraldehyde, propionaldehyde, α-methylpropionaldehyde, 2-methylbutyraldehyde, cyclopentanecarbaldehyde, benzaldehyde, caproaldehyde, carbaldehyde, and the like.

In an preferred embodiment, the methods of the present invention use formaldehyde. The tissue is treated with substantially any amount of formaldehyde that provides the sought after results. The determination of the correct amount of formaldehyde needed for a particular application is well within the abilities of those of skill in the art. For example, a tissue is extracted one or more times with formaldehyde and the extracted material is collected. The amount of infectious material or chemical agent removed by the extraction is preferably determined. When the formaldehyde ceases to remove infectious agent and/or chemical agent from the tissue an end point is reached, which is indicative of the amount of formaldehyde necessary to remove the particular agent from the tissue.

In a preferred embodiment utilizing formaldehyde, the tissue is treated with a formaldehyde solution containing from about 1% to about 10% formaldehyde. The extraction can be performed as a single step. Alternatively, the extraction can be performed as a series of sequential steps. At the end of each sequential step, the formaldehyde containing the extracted agent is preferably removed from the tissue prior to contacting the tissue with a new fraction of formaldehyde.

In another preferred embodiment, the aldehyde used in the method of the present invention is glutaraldehyde. In the same fashion as described above, the tissue can be treated with substantially any amount of formaldehyde that provides the sought after results.

In an exemplary embodiment, the application of glutaraldehyde is similar to that of formaldehyde. The tissue is treated with a solution of glutaraldehyde containing from about 0.2% to about 3% of glutaraldehyde. The extraction can be performed as a single step. Alternatively, the extraction can be performed as a series of sequential steps. At the end of each sequential step, the glutaraldehyde containing the extracted agent is preferably removed from the tissue prior to contacting the tissue with a new fraction of glutaraldehyde.

Although certain preferred embodiments of the present invention are illustrated by the use of formaldehyde or glutaraldehyde to remove infectious agent binding sites from mammalian tissue, this focus on the use of these two aldehydes is for clarity of illustration and should not be construed as defining or limiting the scope of the invention.

The skilled practitioner will recognize that other agents are suitable for use in the method of the present invention. An exemplary agent of use in the invention is a cross linking agent. A list of cross linking agents can be found in U.S. Pat. No. 6,214,054, which is incorporated by reference in its entirety. Yet another exemplary agent is a denaturing agent. Exemplary denaturing agents can be found in U.S. Pat. No. 6,214,054, which is incorporated by reference in its entirety.

In a preferred embodiment, the method of the present invention includes contacting the bioprosthetic tissue with a protic solvent, either alone or in combination with another agent disclosed herein or otherwise known to be useful to remove phospholipids from tissue. Protic solvents of use in practicing the present invention include water, alcohols, carboxylic acids, and the like. Although any protic solvent that has desirable characteristics for a particular application can be used to practice the present invention, certain protic solvents are preferred.

In a preferred embodiment, the method of the present invention uses an alcohol or other solvent incorporating an alcohol. The tissue is treated with substantially any amount of alcohol that provides the sought after results. The determination of the correct amount of alcohol needed for a particular application is well within the abilities of those of skill in the art. For example, a tissue is extracted one or more times with alcohol and the extracted material is collected. The amount of infectious material or chemical agent removed by the extraction is determined. When the alcohol ceases to remove infectious agent and/or chemical agent from the tissue an end point is reached, which is indicative of the amount of alcohol necessary to remove the particular agent from the tissue.

In a preferred embodiment utilizing an alcohol, the tissue is treated with an aqueous alcohol solution containing from about 10% to about 100% alcohol, more preferably from about 20% to about 80% alcohol. The extraction can be performed as a single step. Alternatively, the extraction can be performed as a series of sequential steps. At the end of each sequential step, the alcohol containing the extracted agent is preferably removed from the tissue prior to contacting the tissue with a new fraction of the alcohol.

Preferred alcohols include one or more compounds of the group consisting of methanol, ethyl alcohol, propyl alcohol, butyl alcohol, pentyl alcohol, hexyl alcohol, heptyl alcohol, octyl alcohol, nonyl alcohol, decyl alcohol, and the like. Although any alcohol that has desirable characteristics for a particular application can be used to practice the present invention, certain alcohols are preferred.

In yet another preferred embodiment, a method of the present invention uses ethanol.

In a preferred embodiment, the surfactant is from a group including anionic, cationic, nonionic, and amphoteric surfactants. Although any surfactant that has desirable characteristics for particular application can be used to practice the present invention, certain surfactants are presently preferred.

In another embodiment, the present invention utilizes a surfactant or detergent in the extraction mixture. Any detergent or surfactant known to those of skill in the art is of use in practicing the present invention.

In a preferred embodiment, the method of the present invention uses a nonionic surfactant as the surfactant. Nonionic surfactants include, without limitation, various ethoxylates, carboxylic acid esters, glycol esters, polyoxyethylene esters, anhydrosorbitol esters, ethoxylated anhydrodrosorbitol esters, glycerol esters of fatty acids, carboxylic amides, diethanolamine condensates, and the like. Presently preferred nonionic surfactants also include ethoxylated natural fats, oils and waxes.

In a presently preferred embodiment, the ethoxylated natural fats, oils, and waxes are from a group including, without limitation, lauric, oleic, stearic, and palmitic fatty acids having trade names such as Armotan, Emsorb, Glycosperse, Hodag, and Tween. In a particularly preferred embodiment, the surfactant is Tween 80, an oleic fatty acid.

Of particular interest is a surfactant preparation that can be used to treat tissues that contain or may contain infectious agents. Surfactants of use in the present invention include any surfactant, either substantially pure or containing additives, that prevents or inhibits infectivity in mammalian tissue.

Surfactants such as Tween 80 have been widely used in biochemical applications including: solubilizing proteins, isolating nuclei from cells in culture, growing of tubercule bacilli, and emulsifying and dispersing substances in medicinal and food products. In part, due to these desirable properties, Tween 80 is a presently preferred surfactant.

In a preferred embodiment utilizing Tween 80, the tissue is treated with a solution containing from about to about 0.1% to about 10% Tween 80. The extraction can be performed as a single step. Alternatively, the extraction can be performed as a series of sequential steps. At the end of each sequential step, the Tween 80 containing the extracted agent is preferably removed from the tissue prior to contacting the tissue with a new fraction of Tween 80.

The skilled practitioner will recognize that many other surfactants are suitable for use in the method of the present invention. A list of additional surfactants can be found in U.S. Pat. No. 6,214,054, which is incorporated by reference in its entirety.

In a preferred embodiment, the present invention provides a method for removing essentially all phospholipid in a tissue by contacting the tissue with a combination of formaldehyde, ethanol, and Tween 80. In this embodiment, the binding site for proteins such as prion, acetylcholinesterase, alkaline phosphatase, and Thy-1, are removed. By removing the binding site for the protein, the actual protein can be more easily removed by simple washing or is itself removed as it remains associated with the binding site. In a related embodiment, other chemical combinations able to extract phospholipid would serve an equivalent function.

As discussed above, phospholipids have been implicated as binding sites for prions, infectious agents, and other undesirable substances. A wide variety of phospholipases are known to degrade phospholipids. Thus, in a further embodiment, the method of the present invention removes a binding site from bioprosthetic tissue by contacting the tissue with a preparation including a phospholipase.

The skilled practitioner will appreciate that there are several methods for analyzing the aforementioned phospholipid removal techniques. The tissue can be treated with substantially any amount of phospholipid removing agent that provides the sought after results. The determination of the correct amount of agent is well within the abilities of those of skill in the art. A particularly preferred approach for phospholipid analysis is set out below, in the Examples section.

In yet another preferred embodiment, the method of the present invention further includes one or more steps selected from the group including fixation, bioburden reduction, final sterilization, and packaging. In a related embodiment, the removal step is performed either before, during, or after fixation. In another related embodiment, the removal step is performed during bioburden reduction, sterilization, or packaging. In yet another related embodiment, the method of the present invention includes the removal of the binding site for endogenous prion protein.

In another preferred embodiment, the method also includes a washing step. In still another preferred embodiment, the method includes a terminal sterilization step, such as that described in U.S. Pat. No. 6,214,054.

In yet another preferred embodiment, the structural integrity of the tissue is maintained. Structural integrity can be defined as the ability of tissue to perform it's necessary biological function. The artisan will appreciate that the degree of structural integrity required for the tissue to perform it's necessary function may vary among different types of tissues. Further, particular applications for which the tissue is used may require different levels of structural integrity.

Removing Binding Sites—Protein or Polysaccharide Components

In a second aspect, the present invention provides a method for eliminating or reducing infectivity in a biological material. A related embodiment provides a method for eliminating or reducing infection, including prion infection, in a bioprosthetic tissue. The method includes removing a binding site contained in the tissue so that an infectious agent is prevented or inhibited from binding to the tissue. Typically, the invention is applicable to combating a wide variety of infectious agents, and for preventing their transmission during implantation of a tissue graft. A related embodiment provides a method for removing a protein or polysaccharide component of the binding site to which an infectious agent, including prion protein, can bind. In a further related embodiment, the present invention provides for the removal of a binding site having both a protein and a polysaccharide component, e.g., a proteoglycan.

The invention is also applicable to combating viruses of the family of Picornaviridae, in particular of the genus Hepatovirus, such as the Hepatitis A virus, and for preventing their transmission during tissue grafts. In a this embodiment, the method of the present invention includes removing from a tissue a binding site for the Hepatitis A virus.

Viruses have been implicated in various diseases, including liver disease. In U.S. Pat. No. 5,622,861, Kaplan et al. discuss the Hepatitis A virus, and in particular the isolation of a cellular receptor that is thought to recognize the Hepatitis A virus. Specifically, it is suggested that Hepatitis A virus binds to a 451 amino acid protein. In a preferred embodiment, the present invention provides a method for removing a protein component of a binding site for the Hepatitis A virus.

Subsequent publications by Kaplan and others have further characterized a protein binding domain for the Hepatitis A virus, and a defined amino acid profile has emerged. See, e.g., Thompson et al., “The Cys-Rich Region of Hepatitis A Virus Cellular Receptor 1 is Required for Binding of Hepatitis A Virus and Protective Monoclonal Antibody 190/4,” J. Virol., 72(5): 3751-3761 (1998), Feigelstock et al., “Polymorphisms of the Hepatitis A Virus Cellular Receptor 1 in African Green Monkey Kidney Cells Result in Antigenic Variants That Do No React with Protective Monoclonal Antibody 190/4,” J. Virol., 72(7):6218-6222 (1998), and Silberstein et al., “Neutralization of Hepatitis A Virus (HAV) by an Immunoadhesion Containing the Cysteine-Rich Region of HAV Cellular Receptor-1,” J. Virol, 75(2):717-725 (2001).

In addition to proteins, polysaccharides have been widely implicated as infectious agent binding sites. Infectious agents such as the Sindbis virus, Vaccinia Virus, Classical Swine Fever Virus, Human papillomavirus, Human herpesvirus, Echovirus, Foot and Mouth Disease Virus, and Respiratory Syncytial Virus are believed to recognize a binding site that includes polysaccharide. See, for example, Bymes et al., “Binding of Sindbis Virus to Cell Surface Heparan Sulfate,” J. Virol. 72(9):7349-7356 (1998); Hsiao et al., “Vaccinia Virus Envelope D8L Protein Binds to Cell Surface Chondroitin Sulfate and Mediates the Adsorption of Intracellular Mature Virions to Cells,” J. Virol. 73(10):8750-8761 (1999); Giroglou et al., “Human Papillomavirus Infection Requires Cell Surface Heparan Sulfate,” J. Virol. 75(3):1565-1570 (2001); and Goodfellow et al., “Echoviruses Bind Heparan Sulfate at the Cell Surface,” J. Virol. 75(10):4918-4921 (2001). Polysaccharides are also believed to serve as a binding site for toxigenic molecules. See, e.g., Utt et al., “Helicobacter Pylori Vacuolating Cytotoxin Binding to a Putative Cell Surface Receptor, Heparan Sulfate, Studied by Surface Plasmon Resonance,” FEMS Immunol. Med. Microbiol. 30(2):109-113 (2001).

In a preferred embodiment, the present invention provides a method for removing a binding site from bioprosthetic tissue wherein the binding site includes polysaccharide. Exemplary polysaccharides include, but are not limited to, branched polysaccharides, unbranched polysaccharides, mucopolysaccharides, heteropolysaccharides, and glycosaminoglycans. In a presently preferred embodiment, the method provides for removal of a binding site from the bioprosthetic tissue, where the binding site includes a glycosaminoglycan selected from the group including, without limitation, hyaluronic acid, chondroitin sulfate (A, B, or C), dermatan sulfate, heparan sulfate, heparin (both high and low molecular weight heparin), and keratan sulfate.

Proteoglycans are also known to occur in infectious agent binding sites. Infectious agents such as Orientia tsutsugamushi, human immunodeficiency virus, Niesseria gonorrhoeae, visna virus, and dengue virus are believed to recognize a binding site that includes proteoglycan. See, for example, Ihn et al., “Cellular Invasion of Orientia Tsutsugamushi Requires Initial Interaction with Cell Surface Heparan Sulfate,” Microb. Pathog. 28(4):227-233 (2000); Valenzuela-Femandez et al., “Optimal Inhibition of X4 Isolates by the CXC Chemokine SDF-1a Requires Interaction with Cell-Surface Heparan Sulfate Proteoglycan,” J. Biol. Chem. May 14, 2001 Pubmed epub; Grant et al., “Proteoglycan Receptor Binding by Neisseria Gonorrhoeae MS11 is Determined by the HV-1 Region of OpaA,” Mol. Microbiol. 32(2): 233-242 (1999); and Hilgard et al., “Heparan Sulfate Proteoglycans Initiate Dengue Virus Infection of Hepatocytes,” Hepatology 32(5): 1069-1077 (2000).

In a preferred embodiment, the present invention provides a method for removing a binding site from bioprosthetic tissue wherein the binding site includes proteoglycan. Proteoglycan typically includes a protein core, to which glycosaminoglycan is attached. In this embodiment, exemplary proteoglycans include, but are not limited to, heparan sulfate proteoglycan and chondroitin. Further exemplary proteoglycans will include any of the aforementioned glycosaminoglycans, as well as other glycoproteins that act as a binding site.

Additionally, integrins are known to occur in infectious agent binding sites. Infectious agents such as adenovirus, foot and mouth disease virus, and Streptococcus pyogenes are believed to recognize a binding site that includes integrin. See, for example, Li et al., “Integrin Alpha(v)beta(1) is an Adenovirus Coreceptor,” J. Virol. 75(11):5405-5409 (2001); Miller et al., “Role of the Cytoplasmic Domain of the Beta-Subunit of Integrin Alpha(v)beta(6) in Infection by Foot and Mouth Disease Virus,” J. Virol. 75(9):4158-4164 (2001); and Molinari et al., “Two Distinct Pathways for the Invasion of Streptococcus pyogenes in Non-Phagocytic Cells,” Cell Microbiol. 2(2): 145-154.

In a preferred embodiment, the present invention provides a method for removing a binding site from bioprosthetic tissue where the binding site includes integrin. Integrins typically occur as heterodimers, and include an alpha and a beta subunit. Exemplary integrins include, but are not limited to, alpha(V)beta(1), alpha(6)beta(1), and alpha(L)beta(2). Further exemplary integrins include any heterodimer combination including an alpha integrin subunit, such as alpha(1), alpha(2), alpha(3), alpha(5), alpha(6), alpha(7), alpha(8), alpha(L), alpha(M), alpha(X), alpah(IIB), alpha(V), or alpha(IEL) and a beta subunit, such as beta(1), beta(2), beta(3), or beta(4).

In a further preferred embodiment, the method of the present invention removes a PrP protein binding site from a bioprosthetic tissue. In a particularly preferred embodiment, the binding site may include, without limitation, heparin, heparan sulfate binding protein, integrin, and other cationic domains typically found on cell surfaces or in tissue extracellular matrix. In a related embodiment, the method of the present invention includes contacting the tissue with an enzyme to effect the removal of the binding site. The present invention contemplates that the prion binding site may also include any of the polysaccharides, proteoglycans, or integrins discussed above. In a particularly preferred embodiment, the method uses the enzyme heparinase to digest heparin and thus remove this binding site for the prion protein.

In another preferred embodiment, the method of the present invention removes the PrP binding site by contacting the tissue with a chemical solution that dissociates or extracts the PrP binding site. In a particularly preferred embodiment, the tissue is contacted with a chemical selected from the group including solvents, surfactants, and chaotropic agents. In another preferred embodiment, a polycationic binding site is chemically derivatized, thereby effectively eliminating the binding site for the PrP protein. The artisan will appreciate that the tissue may be contacted with the solution or agent for a period of time sufficient to affect the removal, extraction, or derivatization of the binding site. The present invention further contemplates that this process may optionally include heating, stirring/fluid movement, or both.

The tissue can be treated with substantially any amount of chemical or enzyme solution that provides the sought after results. The determination of the correct amount of chemical or enzyme solution is well within the abilities of those of skill in the art. For example, a tissue is extracted one or more times with the solution, and the amount of specific protein or polysaccharide remaining in the tissue is analyzed. When the amount of remaining protein or polysaccharide ceases to change, an end point is reached, which is indicative of the amount of solution necessary to remove the particular protein or polysaccharide from the bioprosthetic tissue.

In yet another preferred embodiment, the method of the present invention further includes one or more step selected from the group including fixation, bioburden reduction, final sterilization, and packaging. In a related embodiment, the removal step is performed either before, during, or after fixation. In another related embodiment, the removal step is performed during bioburden reduction, sterilization, or packaging. In yet another related embodiment, the method of the present invention includes the removal of binding sites for endogenous prion protein.

Blocking Binding Sites

In a third aspect, the present invention provides a method for blocking a binding site contained in a bioprosthetic tissue. The method includes contacting the tissue with a preparation including a sulfated polyanion, thereby blocking the infectious agent binding site. Alternatively, the method includes contacting the tissue with a preparation including a lipopolyamine, thereby blocking the infectious agent binding site.

During the course of pathogenesis, the infectious agent or toxigenic substance will typically bind to a host cell surface receptor, thereby initiating damage to the cell. In a preferred embodiment, the present invention provides methods for blocking a cell surface receptor, so that these harmful substances are prevented or inhibited from binding to the host cell. Lipoteichoic acid, for example, is produced by the gram positive organism Staphylococcus aureus, and has been strongly implicated in sepsis, a deadly disease. See, for instance, Kengatharan et al., “Mechanism of Gram-Positive Shock: Identification of Peptidoglycan and Lipoteichoic Acid Moieties Essential in the Induction of Nitric Oxide Synthase, Shock, and Multiple Organ Failure,” J. Exp. Med. 188(2):305-315 (1998). The cell surface receptor for lipoteichoic acid also exhibits binding specificity for polyanions such as heparin. See, for example, Dziarski et al., “Heparin, Sulfated Heparinoids, and Lipoteichoic Acids Bind to the 70-kDa Peptidoglycan/Lipopolysaccharide Receptor Protein on Lymphocytes,” J. Biol. Chem. 269(3):2100-2110 (1994).

In a preferred embodiment, the present invention provides a method for blocking an infectious agent binding site in a bioprosthetic tissue by contacting the tissue with a preparation including a sulfonated polyanion. In a preferred embodiment, the sulfonated polyanion is selected from the group including, without limitation, sulfated polysaccharides, polyvinyl sulfate, polyanethole sulfonate, carrageenan, pentosan polysulfate, sulfated colomycin, heparin, heparan sulfate, fucoidan, sulfated cyclodextrins, dextran sulfate, chondroitin sulfate, keratan sulfate, hyaluronic acid, any of the glycosaminoglycans described above, and synthetic variants and analogs thereof. In another embodiment, the preparation includes a lipopolyamine, such as the cationic lipopolyamine DOSPA.

Similarly, sulfated polyanions are known to compete with prion protein by binding to cell surface receptors such as heparan sulfate binding protein, integrins, and other binding domains on cells. When sulfated polyanions are given in cell culture or animal models, for example, binding of prion protein to the cell is prevented and development of spongiform disease symptoms is blocked. Thus, in a related embodiment, the infectious agent is a prion protein. In a further related embodiment, the binding site is a cell surface receptor from the group including, without limitation, heparan sulfate binding protein, integrins, and other binding domains on cells. In another related embodiment, the present invention provides a method for blocking a prion protein binding site in a bioprosthetic tissue by contacting the tissue with a preparation including a sulfated polyanion.

In a related embodiment, the method further includes washing the tissue with repeated washes of a sulfated polyanion, such as a polysulfonated polyglycoside, which competes with the infectious agent for the binding site in the tissue. If washing conditions are sufficient (i.e. compound type and amount, washing conditions and temperature) the washing agent will effectively replace the infectious agent on the binding sites and the PrP can be washed away. Elevated temperatures, such as 37° C. may be desirable in the washing step to promote dissociation-reassociation phenomena and to increase the efficiency of removal of the infectious agent.

The tissue can be treated with substantially any amount of sulfated polyanion that provides the sought after results. The determination of the correct amount of sulfated polyanion needed for a particular application is well within the abilities of those of skill in the art. For example, a tissue is contacted one or more times with sulfated polyanion, and the tissue is then analyzed to determine if additional sulfated polyanion may bind to the tissue. When no more additional sulfated polyanion may bind to the tissue, an end point is reached, which is indicative of the amount of sulfated polyanion necessary to block the particular agent from the tissue. In addition, tissue may be stored in the presence of polyanion in order to ensure saturation of the binding site prior to use.

Removing or Blocking Infectious Agents

In a fourth aspect, the present invention also provides a method for removing or blocking an infectious agent. A related embodiment provides a method for removing or blocking an infectious agent in a biological material. In a particularly preferred embodiment, the method includes contacting the tissue with a substance that binds to the infectious agent. In a further related embodiment, the contacting step may be followed by a washing step.

One of skill in the art will appreciate that the methods of the present invention are well suited for disinfecting or sterilizing a variety of medical instruments or work surfaces. In a preferred embodiment, the present invention includes contacting a medical instrument or a work surface with a substance that binds to the infectious agent, for example, by dipping the instrument in a solution containing the binding substance. This approach may be used to treat pre-existing contamination in an instrument or work surface, and may optionally include a washing step. In a related embodiment, an instrument may be stored in a solution that contains a substance that binds to the infectious agent, thereby preventing or inhibiting contamination of the instrument.

The methods of the present invention may also be used for decontaminating or otherwise treating containers intended for holding or storing biological tissues or medical instruments.

A wide variety of substances are known to bind to infectious agents. Frequently, these substances are investigated as potential therapeutics against infectious agents such as Chlamydia trachomatis, herpes simplex virus, cytomegalovirus, HIV, Malarial sporozoites, and the like. See, for example, Zaretzky et al., “Sulfated Polyanions Block Chlamydia Trachomatis Infection of Cervix-Derived Human Epithelia,” Infect. Immun. 63(9):3520-3526 (1995); Witvrouw et al., “Sulfated Polysaccharides Extracted from Sea Algae as Potential Antiviral Drugs,” Gen. Pharmacol. 29(4):497-511 (1997); Gonzalez et al., “Polysaccharides as Antiviral Agents: Antiviral Activity of Carrageenans,” Antimicrob. Agents Chemother. 31(9):1388-1393 (1987); Gotoh et al., “Sulfated Fibroin, a Novel Sulfated Peptide Derived from Silk, Inhibits Human Immunodeficiency Virus Replication in Vitro,” Biosci. Biotechnol. Biochem. 64(8): 1664-1670 (2000); and Herold et al., “Sulfated Carbohydrate Compounds Prevent Microbial Adherence by Sexually Transmitted Disease Pathogens,” Antimicrob. Agents Chemother. 41(12):2776-2780 (1997).

In a preferred embodiment, the present invention provides a method for blocking an infectious agent from a bioprosthetic tissue by contacting the tissue with a substance that binds to the infectious agent. In a related embodiment, the present invention provides a method for removing an infectious agent from a bioprosthetic tissue by contacting the tissue with a substance that binds to the infectious agent, and thereafter washing the tissue.

Among the myriad infectious agents, prion proteins are of significant interest. Thus, also of interest are substances that bind to prion protein. Among these are sulfated polyanions such as Congo red, heparin, pentosan polysulfate, chondroitin sulfate, and dextran sulfate. See, for example, Caughey et al., “Binding of the Protease-Sensitive Form of Prion Protein PrP to Sulfated Glycosaminoglycan and Congo Red,” J. Virol. 68(4): 2135-2141 (1994); Ehlers et al., “Dextran Sulphate 500 Delays and Presents Mouse Scrapie by Impairment of Agent Replication in Spleen,” J. Gen. Virol. 65: 1325-1330 (1984); Demaimay et al., “Inhibition of Formation of Protease-Resistant Prion Protein by Trypan Blue, Sirius Red and Other Congo Red Analogs,” Arch. Virol. Suppl. 16: 277-283 (2000); Mange et al., “Amphotericin B Inhibits the Generation of the Scrapie Isoform of the Prion Protein in Infected Cultures,” J. Virol. 74(7): 3135-3140 (2000); Caughey, “Scrapie-Associated PrP Accumulation and Agent Replication: Effects of Sulphated Glycosaminoglycan Analogues,” Philos. Trans. R. Soc. Lond. B. Biol. Sci. 343(1306): 399-404 (1994); Priola et al., “Porphyrin and Phthalocyanine Antiscrapie Compounds,” Science 287: 1503-1506 (2000); and Tagliavini et al., “Effectiveness of Anthracycline Against Experimental Prion Disease in Syrian Hamsters,” Science 276: 1119-1122 (1997).

The etiology of prion disease is unique, and is believed to involve a recruitment process whereby the infectious form of the prion protein facilitates the conversion of normal prion into that of infectious prion. Due to this remarkable mechanism, the infectious agent blocking substance used in the present invention includes any substance that blocks the prion protein in its infectious form, thereby inhibiting or eliminating the ability of the infectious form to further transform non-infectious prion into the infectious form. Equally as important, the infectious agent blocking substance will also include any substance which blocks the prion protein in its non-infectious prion, and hinders or otherwise prevents the transformation of the non-infectious prion into the infectious form.

In a particularly preferred embodiment, the present invention provides a method for blocking an infectious agent in a bioprosthetic tissue by contacting the tissue with preparation including polysaccharide. Exemplary polysaccharides include, but are not limited to, branched polysaccharide, unbranched polysaccharide, mucopolysaccharide, heteropolysaccharide, and glycosaminoglycan. In a presently preferred embodiment, the preparation includes a glycosaminoglycan selected from the group including, without limitation, hyaluronic acid, chondroitin sulfate (A, B, or C), dermatan sulfate, heparan sulfate, pentosan polysulfate, heparin (both high and low molecular weight heparin), keratan sulfate, and glycosaminoglycan analogs. The artisan will appreciate that glycosaminoglycan will also include synthetic variants and analogs thereof. For example, Congo Red is a recognized glycosaminoglycan analog. Accordingly, the preparation may include Trypan Blue, Sirius Red F3B, Evans Blue, Fast Red, Trypan Red, Primuline, Thioflavin-S, or the like.

In a further preferred embodiment, the method includes contacting the tissue with a preparation including heteropolyanion, polyene antibiotic, polyanion, sulfated polyanion, sulfated cyclodextrin, carrageenan, and sulfated polysaccharide.

In another preferred embodiment, the present invention provides a method for blocking an infectious agent in a bioprosthetic tissue by contacting the tissue with a preparation including an antifungal agent. In a related embodiment, the preparation includes a polyene antibiotic, such as Amphotericin B. Further, the preparation may include a polyanionic antifungal agent, or other compounds used to treat or diagnose amyloid disease.

In a preferred embodiment, the preparation includes a porphoryin or a phthalocyanine. Exemplary compounds of this type include PcTS (phthalocyanine tetrasulfonate), TMPP-Fe³⁺ [meso-tetra(4-N-methylpyridyl)porphine iron (III)], DPG2-Fe³⁺ [deuteroporphryin IX 2,4-bis-ethylene glycol) iron(III)], or other tetrasubstituted porphoryin.

In a further embodiment, the present invention provides a method for blocking an infectious agent in a bioprosthetic tissue by contacting the tissue with a preparation including an anthracycline, such as 4′-iodo-4′-deoxy-doxorubicin. In another embodiment, the method involves contacting a tissue with a preparation including sulfated fibroin, a peptide derived from silk. Optionally, the method may contacting a tissue with a preparation including a sulfated carbohydrate, or a sulfated maltoheptaose derivative, such as N-acetyl- [symbol] -maltoheptaosylamine sulfate.

In an alternate embodiment, the present invention provides a method for blocking an infectious agent in a bioprosthetic tissue by contacting the tissue with a preparation including synthetic sulfated polymer, such as a copolymer of acrylic acid with vinyl alcohol sulfate (PAVAS). In another embodiment, the preparation includes a sulfated chaotropic surfactant, such as sodium laurel sulfate.

In another preferred embodiment, the present invention provides a method for blocking an infectious agent in a bioprosthetic tissue by contacting the tissue with a preparation including a branched polyamine. Exemplary branched polyamines include, without limitation, polyamidoamine and polypropyleneimine (PPI) dendrimers, polyamindoamide dendrimers, and polyethyeleneimine. In a related embodiment, the preparation includes branched polyamine and chloroquine.

In a presently preferred embodiment, the present invention provides a method for blocking an infectious agent in a bioprosthetic tissue by contacting the tissue with a preparation including a lysosomotropic agent or a cysteine protease inhibitor. Exemplary lysosomotropic agents include, without limitation, quinacrine, tilorone, chloroquine, and suramine. Exemplary cysteine protease inhibitors include, without limitation, E-64d, E-64, and leupeptin.

In another preferred embodiment, the present invention provides a method for blocking an infectious agent in a bioprosthetic tissue by contacting the tissue with a preparation including a denaturing agent such as glutaraldehyde. While no theoretical explanation can be given with certainty for the blocking effect of this denaturing agent, it is believed that it prevents infectious prion replication by stabilizing the non-infectious form of the prion protein, and in particular by modifying the lysine residues at positions 184 and 193 of the prion. The artisan will recognize that other substances that similarly modify these lysine residues, or otherwise stabilize the non-infectious form of the prion protein, are well suited for use in the method of the present invention.

In yet another preferred embodiment, the present invention provides a method for blocking an infectious agent in a bioprosthetic tissue by contacting the tissue with a beta-sheet blocker, or a beta sheet breaker peptide, such as iPrP13. This approach is based on the concept that the secondary structure of the infectious form of the prion protein presents beta sheet conformation, whereas the non-infectious form contains alpha helix.

While the above embodiments envisage blocking the infectious agent, it is also apparent that by contacting the tissue with copious amounts of the substance that binds to the infectious agent, bioprosthetic tissue can be rendered free of some or all contamination.

The artisan will appreciate that these binding substances may be used to bind either to exogenous prion protein, or to endogenous prion protein. Similarly, the binding substances may be used to disinfect contaminated or infected tissue, or to prohibit tissue from becoming contaminated or infected. In a preferred embodiment, the tissue is perfused with a preparation including a binding substance, thereby blocking any prion infectivity. In a related preferred embodiment, the present invention provides a method for preventing or inhibiting infectivity in a bioprosthetic tissue by contacting the tissue with one or more of the above mentioned binding substances, and extracting and washing away the bound complex of binding substance and prion. In these embodiments, any possible conversion of PrP^{C} to the mutant form, PrP^{Sc} is prevented or inhibited.

The tissue can be treated with substantially any amount of infectious agent blocking substance that provides the sought after results. The determination of the correct amount of blocking substance is well within the abilities of those of skill in the art. For example, a tissue is extracted one or more times with the blocking substance, and the amount of infectious agent remaining in the tissue is analyzed. When the amount of remaining infectious agent ceases to change, an end point is reached, which is indicative of the amount of substance necessary to block the particular infectious agent from the bioprosthetic tissue.

The materials, methods and devices of the present invention are further illustrated by the examples that follow. These examples are offered to illustrate, but not to limit the claimed invention.

### EXAMPLES

### Example 1

1.1 Phospholipid Removal

Several lots of fresh pericardial tissue were obtained from two different vendors. The samples were subjected to the following protocol: (1) a first fixation step, (2) a second fixation step, (3) a first bioburden reduction process, (4) a second bioburden reduction process, and (5) a terminal liquidation sterilization process.

1.2 Phospholipid Analysis

Studies were performed to objectively assess the efficacy of the above-described method in removing phospholipid from tissue. The samples were smashed, lyophilized, weighed, rehydrated with water, and extracted with a mixture including chloroform, methanol, and BHT. Saline was added to the mixture, and the sample was centrifuged. The lower phase of chloroform was collected and dried under nitrogen gas, and then reconstituted with a mixture of chloroform and BHT.

The samples were then spotted on a thin layer chromatography (TLC) plate with mixed phospholipid standards. The plates were dried, stained, and scanned to measure the density of fluorescence spots. The final results were converted from 0 g phospholipid in the spot to 0 g/mg dry weight tissue.

1.3 Results

The following phospholipid levels were observed following each step of the protocol.

| | Average | Total | |
|---|---|---|---|
| | Percent | Phospholipid | |
| Protocol Step | Removal | \overline{X} ± SD | Range |
| Fresh sample | (N/A) | 7.244 ± 3.055 | (2.180-12.469) |
| First fixation step | (46%) | 3.884 ± 1.612 | (1.887-7.552) |
| Second fixation step | (34%) | 4.775 ± 1.326 | (2.929-7.722) |
| First bioburden reduction | (76%) | 0.553 ± 0.225 | (0.248-0.983) |
| Second bioburden reduction | (98%) | 0.132 ± 0.057 | (0.061-0.302) |
| Terminal liquidation | (98%) | 0.120 ± 0.084 | (0.026-0.326) |
| sterilization | | | |

The percentage of phospholipid (PL) removal was determined as follows.

$$\% PL Removal = \frac{Total PL Fresh Tissue - Total PL Processed Tissue}{Total PL Fresh Tissue}$$

As illustrated by the formula, the percentage of phospholipid removal was calculated in comparison to fresh tissue.

It is understood that the examples and embodiments described herein are for illustrative purposes only and that various modifications or changes in light thereof will be suggested to persons skilled in the art and are to included within the spirit and purview of this application and are considered within the scope of the appended claims. All publications, patents, and patent applications cited herein are hereby incorporated by reference in their entirety for all purposes.

1. A method of eliminating or reducing infection in a bioprosthetic tissue, the method comprising blocking a binding site contained in the tissue so that an infectious agent is prevented or inhibited from binding to the binding site.

2. The method of claim 1, wherein the infection is a prion infection, and the infectious agent is prion protein.

3. The method of claim 1, wherein the structural integrity of the tissue is maintained.

4. The method of claim 1, wherein the blocking step further comprises contacting the bioprosthetic tissue with a preparation comprising one or more polysulfonated polyglycosides.

5. The method of claim 4, wherein the one or more polysulfonated polyglycosides are selected from a group consisting of pentosan polysulfate, sulfated colomycin, dextran sulfate, sulfated carageenans, and heparin/heparan sulfate.

6. The method of claim 4, wherein the contacting step is performed at a temperature of about 37° C.

7. The method of claim 1, wherein the contacting step promotes the dissociation of prion protein from the bioprosthetic tissue.
